(19) United States
(12) Patent Application Publication
(10) Pub. No.: US 2020/0113487 A1
(43) Pub. Date: Apr. 16, 2020

(54) PATIENT MONITORING SYSTEM

(71) Applicant: Hello Nurse Medical Innovation, Inc., Irvine, CA (US)

(72) Inventor: Sharon Lin Charna, Irvine, CA (US)

(21) Appl. No.: 16/713,919

(22) Filed: Dec. 13, 2019

Related U.S. Application Data
(63) Continuation-in-part of application No. 16/006,898, filed on Jun. 13, 2018, now Pat. No. 10,517,511.
(60) Provisional application No. 62/849,501, filed on May 17, 2019.
(60) Provisional application No. 62/524,860, filed on Jun. 26, 2017.

(30) Foreign Application Priority Data
Jun. 14, 2017 (TW) 106208622
Jul. 28, 2017 (TW) 106211104

(51) Int. Cl. A61B 5/11; A61B 5/00

(52) U.S. Cl. CPC A61B 5/1115; A61B 5/747; A61B 2562/0247; A61B 5/4809; A61B 5/746; A61B 5/0004

(57) ABSTRACT

A patient monitoring system is presented. Pressure change indications from a pressure-sensitive sensor pad are transmitted to a monitoring alert apparatus as patient status signals. The monitoring alert apparatus uses the received patient status signals to determine one or more patient statuses, which may include sleep safety assessment, durations of pressure contacts, blood oxygen level, breathing rates, heart rates, blood pressure, brief wetness, etc. The sleep safety assessment and the duration of pressure contacts are determined based on pressure changes detected at different pressure-sensitive sensing zones of the pressure-sensitive sensor pad. The monitoring alert apparatus determines whether to generate one or more alerts based on the determined patient statuses.

### CLAIM OF BENEFIT TO PRIOR APPLICATIONS

This application claims the benefit of U.S. provisional application No. 62/849,501, filed on May 17, 2019, titled “Wireless Patient Monitoring System.” This application is also a continuation-in-part application of U.S. patent application Ser. No. 16/006,898, filed on Jun. 13, 2018, titled “Patent Monitoring System”. U.S. patent application Ser. No. 16/006,898 claims the benefit of U.S. provisional patent application No. 62/524,860 filed on Jun. 26, 2017, Taiwanese patent application No. 106208622 filed on Jun. 14, 2017, and Taiwanese patent application No. 106211104 filed on Jul. 28, 2017. All the above are hereby incorporated by reference.

### BACKGROUND

### Technical Field

The present invention wholly relates to the technical field of medical care facilities, and in particular, to a notification system that can be laid flat on a proper position of a sickbed to know patient's statuses in the bed at any time by the body weight of a patient; and to predict and judge, according to the patient's statuses in the bed, whether the patient intends to get out of bed.

### Background

As medical techniques and technologies dramatically develop, human lifespan is prolonged. However, while the lifespan is prolonged, the chance of illness also increases significantly. Although modern medicine can overcome most diseases and can give patients healthy bodies, the patients must eventually undergo medical processes before recovering.

In facing relatively serious or complicated illness conditions, some patients have to be on bed rest to receive medical treatment or observation, and definitely, a few patients have difficulty in getting up alone or even are unable to do so. Further, there also exist a minority of patients who are able to take care of themselves but still need assistance or observation from nursing staff or medical staff. Therefore, the workload of the nursing staff or medical staff becomes increasing heavy. Moreover, from the perspective of an increasingly extensive and long-lasting course, use of only technological medical equipment fails to effectively reduce the demands for the number and the workload of the nursing staff.

It has been pointed out by related follow-up study in Taiwan that, the ratio of patients to nurses in Taiwan's regional or above hospitals is 12:1 during the years from 2006 to 2007. Moreover, due to the social pattern characterized by rapid population aging, it can be predicted that there will be increasingly more bedridden patients and the time cost in the sickbed also significantly increases. Therefore, the demands for the number and the workload of nursing staff also significantly grow. During the care of mentally retarded patients who act freely but have confused consciousness, or patients who have physical and mental dysfunction and require caregivers' accompaniment, the caregiver always worries that the patient leaves the sickbed alone or drops from the sickbed to cause accidental damage. When the patient drops from the sickbed or tumbles after leaving the sickbed, an adverse consequence such as severe harm or even death may be incurred if the caregiver fails to immediately handle such situations in the first place.

Taiwan utility model patent (certification NO.: M465634) No. 102207861 that has been published discloses “Protective Alert device for Sickbed”, the sickbed having a bed surface for supporting the human body, a front end portion closed to the head of the human body, and a rear end portion closed to the feet. The protective alert device includes: a carrier configured on the sickbed; a light sensitive unit, mounted on the carrier and forming a height difference with the bed surface of the sickbed, and used to receive light rays along a sickbed short-side direction within a sensing range not larger than the length of the sickbed short side; and an alarm unit, electrically connected to the light sensitive unit and used to generate a warning signal when the light rays in the sensing range have a change.

In the foregoing first former invention, limited to the sickbed short-side direction, the sensing range can be extended to the whole sickbed with reduced dead angles, thus achieving a protection effect. However, this former invention cannot sense patient's statuses in bed, and further cannot predict and judge whether the patient intends to get out of bed. When the patient drops from the sickbed or tumbles after leaving the bed, an adverse consequence such as severe harm or even death can be avoided if a caregiver makes prevention in advance. Taiwan utility model patent (certification NO.: M447769) No. 101219854 that has been published discloses “Sickbed for Sensing Patient's Action of Leaving Bed”, where the sickbed includes: a bedstead having at least one off-bed side; a detection module including a plurality of detection units arranged on the bedstead, each detection unit being able to send out a warning signal when sensing that the patient's body passes through the off-bed side; a processing module, connected to the sensing units of the sensing module via a signal and capable of receiving and processing the warning signals sent by the detection units, the processing module further sending out an off-bed signal when simultaneously receiving multiple warning signals; and an alert module, connected to the processing module via a signal and capable of sending out an alert signal when driven by the off-bed signal.

In the foregoing second former invention, the detection units can send out a warning signal when the patient leaves the bed, such that the nursing staff are timely informed and then immediately go to the ward to take care of the patient. However, this former invention cannot sense patient's statuses in bed, and further cannot predict and judge whether the patient intends to get out of bed. When the patient drops from the sickbed or tumbles after leaving the bed, an adverse consequence such as severe harm or even death can be avoided if the caregiver makes prevention in advance.

Therefore, it is necessary to solve the foregoing problems so as to avoid accidents and reduce the manpower, costs, and time. In view of the defects in the prior art, the applicants finally conceive the present invention through careful experimentation and research with perseverance, to overcome the defects in the prior art.

### SUMMARY OF THE INVENTION

The present invention provides a patient monitoring system. Pressure change indications from a pressure-sensitive sensor pad are transmitted to a monitoring alert apparatus as patient status signals. The monitoring alert apparatus uses the received patient status signals to determine one or more patient statuses, which may include sleep safety assessment, durations of pressure contacts, blood oxygen level, breathing rates, heart rates, blood pressure, brief wetness, etc. The sleep safety assessment and the duration of pressure contacts are determined based on pressure changes detected at different pressure-sensitive sensing zones of the pressure-sensitive sensor pad. The monitoring alert apparatus determines whether to generate one or more alerts based on the determined patient statuses.

### BRIEF DESCRIPTION OF THE DRAWINGS

FIG. 1 is a schematic architecture diagram of an embodiment of a patient monitoring system according to the present invention.

FIG. 2 is a schematic architecture diagram of another embodiment of a patient monitoring system according to the present invention.

FIG. 3 is a schematic architecture diagram of another embodiment of a patient monitoring system according to the present invention.

FIG. 4 is a schematic structural diagram of a section of a pressure-sensitive sensor pad according to the present invention.

FIG. 5 is a schematic diagram showing a distribution manner of pressure-sensitive sensing zones according to the present invention.

FIG. 6 illustrates an example display of various patient statuses generated by the patient monitoring system.

FIG. 7 is a schematic flowchart of operations of a patient monitoring system according to the present invention.

FIG. 8 conceptually illustrates an electronic system with which some embodiments of the present disclosure are implemented.

### DETAILED DESCRIPTION

Technical means used by the present invention to achieve the set invention objectives is further described below with reference to the accompanying drawings and preferred embodiments of the present invention.

In some embodiments, the patient monitoring system is a patient off-bed notification system that sends out a signal to notify a caregiver before a patient leaves the bed, and continuously send out a notification signal after the patient leaves the bed, so as to remind the caregiver to go to the ward and take care of the patient. Thus, the harm caused when the patient leaves the bed alone can be minimized, thus effectively improving the work efficiency of the caregiver.

The patient monitoring system includes a pressure-sensitive sensor pad, a signal converter, and a signal transmitter. The pressure-sensitive sensor pad includes an upper conductive layer, a bottom conductive layer and an insulating layer arranged therebetween, where the upper conductive layer and the bottom conductive layer can generate an electrical signal due to a pressure change. The signal converter is electrically connected to the pressure-sensitive sensor pad, and is used to receive the electrical signal sent by the pressure-sensitive sensor pad and perform operational analysis to produce a corresponding patient status signal. The signal transmitter is electrically connected to the signal converter and transmits the signal.

The pressure-sensitive sensor pad can be further divided into at least one or a plurality of pressure-sensitive sensing zones, where the at least one or the plurality of pressure-sensitive sensing zones produces electrical signals due to a foreign force or pressure change, the electrical signals being the same or varying from each other, or some of them being the same.

The number of the at least one or the plurality of pressure-sensitive sensing zones and their positions are not limited, and can be determined according to sensed pressures and positions. For example, the pressure-sensitive sensor pad is divided into three zones: a left one, a middle one and a right one, and further the middle zone thereof is divided into four sub-zones; in addition, these pressure-sensitive sensing zones have the same or different sensing areas, or some of them have the same sensing areas.

In some embodiments, the insulating layer is provided with multiple pierced regions used to form these pressure-sensitive sensing zones by partitioning.

In some embodiments, the system further includes a server, used to receive the patient status signal sent by the signal transmitted. The server may further include a display unit, used to store and/or display information corresponding to the patient status signal, where the display unit is a computer screen or an LED panel. For example, the display unit can display whether the patient is in a normal status or a dangerous status, and also display patient information including the ward number, bed number, patient name, occurrence time of a dangerous condition, and the like.

The patient monitoring system may further include at least one alert device for sending out a warning signal, where the alert device is communicatively connected to the server and used to receive the information corresponding to the patient status signal. When the patient is in a dangerous status, the display unit can display the patient information including the ward number, bed number, patient name, occurrence time of a dangerous condition, and the like; and the alert device can synchronously send out a warning signal. The warning signal may be presented in at least one of the following manners: sound, an image, vibration, light, and a digital signal. The alert device may be at least one of a wireless BB call, a mobile phone, a buzzer, an alarm lamp, and an audio alarm; or may also be an alert device having alarm functions of at least two of a buzzer, an alarm lamp, and an audio alarm. For example, the alert device can display different lamp signals and emit warning sound to indicate the status of the patient; or a mobile phone is used to emit warning sound and display patient information including the ward number, bed number, patient name, occurrence time of a dangerous condition, and the like.

When the patient is in a dangerous status, the alert device can unceasingly send out the warning signal at regular intervals, till the warning is lifted. The signal transmitter can transmit a signal via a wireless transmission manner such as WIFI, ZigBee, or Bluetooth. The pressure-sensitive sensor pad is arranged in a bed, where the bed further includes an upper cushion and a bottom cushion, and the pressure-sensitive sensor pad can be placed on the upper cushion or between the upper cushion and the bottom cushion.

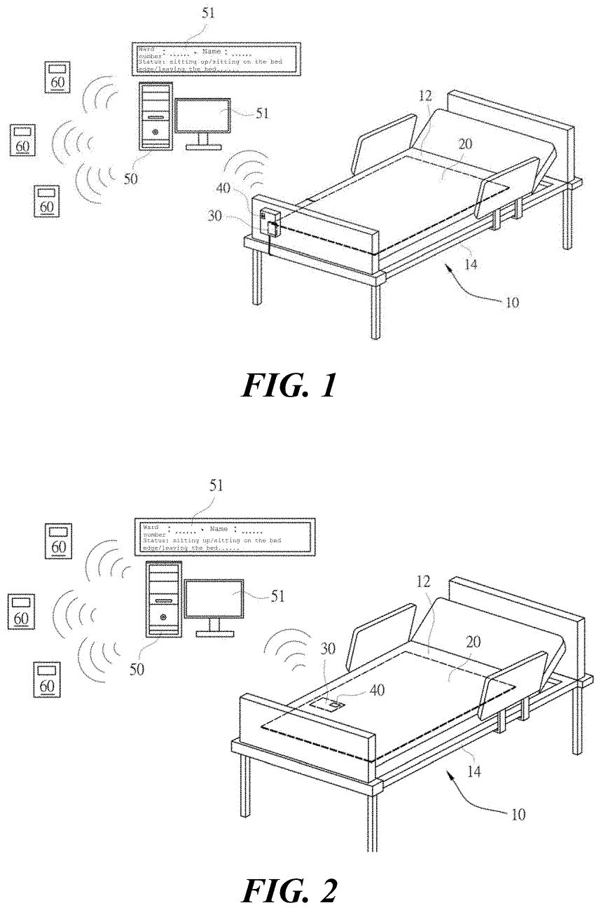

Refer to FIG. 1 and FIG. 2, which are schematic diagrams respectively showing a first embodiment and a second embodiment of an architecture of a patient monitoring system of the present invention. As illustrated, the patient monitoring system includes a pressure-sensitive sensor pad (20), a signal converter (30), and a signal transmitter (40) that are arranged in a bed (10). The bed (10) includes an upper cushion (12) and a bottom cushion (14). The bottom cushion (14) is mainly used as a support, and therefore may be a part of a bedstead (not shown in the figures) in practice. The signal converter (30) and the signal transmitter (40) are arranged outside, and are connected to the pressure-sensitive sensor pad (20) via an information transmission line, as shown in FIG. 1.

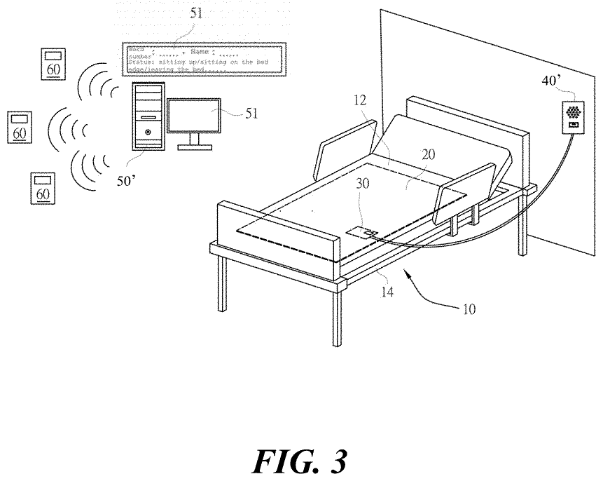

Alternatively, the signal converter (30) and the signal transmitter (40) are integrated in the pressure-sensitive sensor pad (20), as shown in FIG. 2. The present invention does not limit an arrangement manner thereof. Furthermore, the patient monitoring system of the present invention can also be connected with calling system of the hospital nurse station directly. Refer to FIG. 3, which is schematic diagram showing another embodiment of an architecture of a patient monitoring system of the present invention. In the embodiment, the signal transmitter is an existing calling device (40′) in the ward, and the server is the monitoring alert apparatus (50′) set at the hospital nurse station. The signal converter (30) is electrically connected to the calling device (40′) and sends the patient status signal to the monitoring alert apparatus (50′) by the calling device (40′).

In some embodiments, the calling device (40′) is installed on the wall surface near the sickbed and is the existing one which has been installed in the ward. In addition, the calling device (40′) has a communication interface that can be respectively connected to the signal converter (30) and the monitoring alert apparatus (50′) set at the nursing station in a manner of augmenting the communication interface with wire connection or wireless connection. The calling device (40′) is provided with an emergency button. When an emergency occurs to the patient, the patient may press the emergency button to send a warning signal to the monitoring alert apparatus (50′). And when the signal converter (30) is electrically connected to the calling device (40′), the calling device (40′) can also send the patient status signal from the signal converter (30) to the monitoring alert apparatus (50′) to notify the nursing staff if the patient is about to leave the bed, about to fall, in need of repositioning (to prevent bedsores), etc.

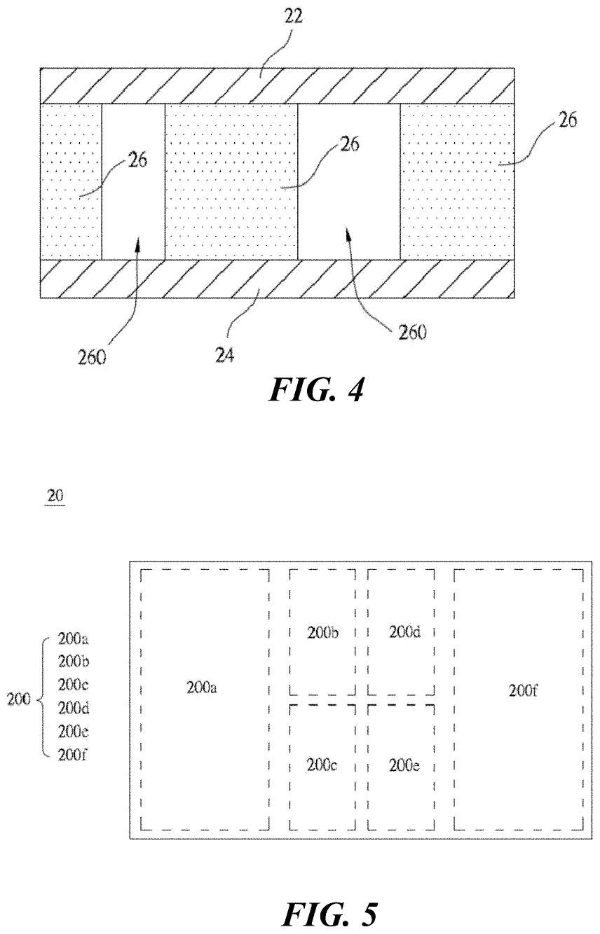

Refer to FIG. 4, which is a schematic structural diagram of a section of a pressure-sensitive sensor pad according to the present invention. The pressure-sensitive sensor pad (20) is designed into a rectangular sheet according to the shape of the bed (10). The pressure-sensitive sensor pad (20) may be a soft pad formed by an upper conductive layer (22), a bottom conductive layer (24), and an insulating layer (26) arranged therebetween, where three layers are arranged in parallel with each other. The insulating layer (26) is provided with multiple pierced regions (260). These pierced regions (260) partition the upper conductive layer (22) and the bottom conductive layer (24) into multiple pressure-sensitive sensing zones (200). Each pressure-sensitive sensing zone (200) forms a pressure-resistant pressure sensor structure, and these pressure-resistant pressure sensor structures are separately electrically connected to the signal converter (30). In other words, the pressure-sensitive sensor pad (20) is a pad or mattress that is populated with pressure sensors that are implemented by the pressure-sensitive sensing zones (200).

Refer to FIG. 5, which is a schematic diagram showing a distribution manner of the pressure-sensitive sensing zones of the pressure-sensitive sensor pad according to the present invention. The pressure-sensitive sensing zones (200) are distributed on different positions of the pressure-sensitive sensor pad (20) according to arrangement positions of these pierced regions (260). The pressure-sensitive sensing zones are at least located on the left, middle, and right positions of the pressure-sensitive sensor pad (20), and there exist at least three pressure-sensitive sensing zones (200). According to an embodiment of the present invention, the pressure-sensitive sensing zones (200) may be distributed on the left, middle, and right positions of the pressure-sensitive sensor pad (20) to form six pressure-sensitive sensing zones (200a, 200b, 200c, 200d, 200e, 200f). These pressure-sensitive sensing zones (200) may have the same or different areas, or some of them have the same areas. According to an embodiment of the present invention, the left and right pressure-sensitive sensing zones (200a and 200f) have roughly the same areas, while the four pressure-sensitive sensing zones (200b, 200c, 200d, 200e) distributed in the middle have roughly the same areas. It should be noted that, FIG. 5 only show a preferred embodiment of the pressure-sensitive sensing zones (200) of the present invention, but is not intended to limit their distribution positions, relative positions, and area size.

Furthermore, the pressure-sensitive sensor pad (20) can be placed on the upper cushion or between the upper cushion (12) and the bottom cushion (14). The present invention does not particularly limit the size of the pressure-sensitive sensor pad (20), and the size thereof can be designed or adjusted according to the body type of the patient or the size of the sickbed. In addition, the magnitudes of pressure applied by the patient on the pressure-sensitive sensing zones (200), and pressed parts vary according to whether the patient lies flat or sits on the bed, and/or according to different body types of the patients. In consideration of these facts, the pressure-sensitive sensor pad (20) is designed to have different sizes with reference to the magnitudes of pressure applied on the pressure-sensitive sensing zones (200) and corresponding positions of the pressed parts. For example, in an embodiment of the present invention, a pressure-sensitive sensor pad of 170 cm×90 cm (length×width) may be used, but the present invention is not limited thereto; and a pressure-sensitive sensor pad (20) of another size may also be used. In some embodiments, the pressure-sensitive sensor pad (20) is further equipped with an air pump to generate alternate pressure to the patient in order to minimize prolonged pressure contact.

As described above, for the pressure-sensitive sensor pad (20) that is used for sensing the pressure in a pressure-resistant manner and formed by the upper conductive layer (22), the bottom conductive layer (24), and the insulating layer (26), when the insulating layer (26) deforms under stress, the resistance between the insulating layer (26) and the conductive layers (22 and 24) changes. As such, an electrical signal having a certain relationship with the voltage can be output through a measurement circuit.

The signal converter (30) may be formed by an integrated circuit board in which an operation processing chip is embedded. The signal converter is used to collect electrical signals (analog signals) from the pressure-sensitive sensing zones (200) of the pressure-sensitive sensor pad (20); and perform operation processing, analysis, and interpretation to generate one or more patient status signals. Then, the signal transmitter (40) or the calling device (40′) (for example, ZigBee wireless transmission technology with rather low power consumption) sends the one or more patient status signals to a server (50) (for example, a host computer in a hospital nurse station, a desktop computer for home-based care, or a mobile device such as a laptop, a tablet computer or a smart phone) or the monitoring alert apparatus (50′). The server (50) or the monitoring alert apparatus (50′) can interpret these signals, record them, and display them on a display unit (51). The display unit (51) may be a computer screen of the host computer or a display screen of a mobile device; or may also be an LED panel connected to the server (50) or the monitoring alert apparatus (50′). In addition, the display unit (51) can also display information that corresponds to a warning signal and includes the position of the bed (10) and symptoms or name of the patient using the bed (10).

When the patient is in a dangerous status, various alert devices (60) connected to the server (50) or the monitoring alert apparatus (50′) via a wireless transmission technology such as WIFI, ZigBee, or Bluetooth are used to receive a signal timely sent by the server (50) or the monitoring alert apparatus (50′) and display corresponding information, such that a caregiver can observe the status of the patient at any time. These alert devices (60) display information in different and appropriate visual, auditory, or tactile manners such as light, sound or vibration. For example, the warning signal can be presented in at least one of the following manners: sound, an image, vibration, light, and a digital signal. The alert device may be at least one of a wireless BB call, a mobile phone, a buzzer, an alarm lamp, and an audio alarm; or may also be an alert device having alarm functions of at least two of a buzzer, an alarm lamp, and an audio alarm. Moreover, the server may also display the warning signal on a mobile phone of the caregiver synchronously, such that the caregiver is informed and immediately goes to the ward to find out the patient's status. In some embodiments, a mobile device and its display unit may simultaneously serve as an alert device 60 and the server 50 (or the monitoring alert apparatus 50′).

In some embodiments, whether a patient is in a dangerous status is in part determined based on the patient's position on the bed (10), e.g., whether the patient is lying flat, sitting up, lying down, or lying near the bed edge. The following Table 1 illustrates patient positions on the bed (10) that are detected according to the distribution of the pressure-sensitive sensing zones shown in FIG. 4. In this embodiment, the pressure-sensitive sensing zone 200a is at the position near the head of a patient, and the pressure-sensitive sensing zone 200f is at the position near the feet of the patient.

TABLE 1

| | Pressure conditions in | | | | | |
|---|---|---|---|---|---|---|
| Patient Positions | different pressure-sensitive sensing zones | | | | | |
| On the Bed | 200a | 200b | 200c | 200d | 200e | 200f |
| Sit up | | ✓ | ✓ | | | |
| Sit up | | | | ✓ | ✓ | |
| Sit up | | | | ✓ | ✓ | ✓ |
| Dangerous | | ✓ | | | | |
| Dangerous | | | ✓ | | | |
| Dangerous | | | | ✓ | | |
| Dangerous | | | | | ✓ | |
| Dangerous | | | | | | ✓ |
| Dangerous | | ✓ | | ✓ | | |
| Dangerous | | | ✓ | | ✓ | |
| Dangerous | | | | ✓ | | ✓ |
| Dangerous | | | | | ✓ | ✓ |
| Sleep (Lying flat) | ✓ | ✓ | ✓ | | | |
| Lie on the bed edge | ✓ | ✓ | | | | |
| Lie on the bed edge | ✓ | | ✓ | | | |

Multiple pressure sensors formed by the pressure-sensitive sensing zones (200) can sense pressure according to whether there is an external pressure applied thereon, that is, detecting pressure contacts between the patient's body and the pressure-sensitive sensor pad (20) according to the statuses of the patient lying thereon. When the pressure on some of the pressure-sensitive sensing zones (200) increases, the impedance between the upper conductive layer (22) and the bottom conductive layer (24) is reduced and thus the capacitance is changed. Then, the signal transmitter (40) or the calling device (40′) sends out an appropriate ZigBee wireless communication signal or wire communication signal. Upon the pressure-sensitive sensing zones (200) sending out corresponding signals according to whether there is a pressure (that is, the weight of the patient) applied thereon, the server (50) or the monitoring alert apparatus (50′) determines whether the patient is lying flat, turning over, lying on his/her side, sitting up, or sitting on the bed edge. The server (50) or the monitoring alert apparatus (50′) may also determine whether the patient intends to leave the bed and whether the leaving time is abnormal. Then, the server sends out a corresponding signal or warning according to a determination result such that a user of the alert device can immediately obtain first-hand information, where the information may include the ward number, bed number, patient name, occurrence time of a dangerous condition, and the like.

In some embodiments, the alert device (60) has an alarm having buzzing and lamp warning functions. For example, when the pressure-sensitive sensing zones 200a, 200b and 200c simultaneously sense the pressure, the server (50) or the monitoring alert apparatus (50′) determines, according to a patient status signal, that the patient is in a normal flat-lying status. Therefore, it is unnecessary to send out a warning signal, and in this case, the alert device shows a green lamp to indicate a normal status. When the pressure-sensitive sensing zones 200a and 200b simultaneously sense the pressure, the server (50) or the monitoring alert apparatus (50′) determines, according to a patient status signal, that the patient is in a dangerous status of lying near the bed edge or sitting on the bed edge. Therefore, the server sends a warning signal to the alert device, and displays patient information on an LED panel of the nurse station. In this case, the alert device shows a warning yellow lamp and emits buzz sound, to indicate that the patient is currently in a dangerous status of lying near the bed edge. After receiving the signal, nursing staff can timely go to the ward to handle the situation. Likewise, when the pressure-sensitive sensing zones 200a and 200c simultaneously sense the pressure, it also indicates that the patient is currently in a dangerous status of lying near the bed edge.

When the pressure-sensitive sensing zones 200b and 200c simultaneously sense the pressure, the server (50) or the monitoring alert apparatus (50′) may determine, according to a patient status signal, that the patient is in a dangerous status of sitting on the bed. Therefore, the server sends a warning signal to the alert device, and displays patient information on an LED panel of the nurse station. In this case, the alert device shows a warning blue lamp and emits buzz sound, to indicate that the patient is currently in a dangerous status of sitting on the bed. After receiving the signal, nursing staff can timely go to the ward to handle the situation. Likewise, when the pressure-sensitive sensing zones 200d and 200e, or the pressure-sensitive sensing zones 200d, 200e and 200f simultaneously sense the pressure, it also indicates that the patient is currently in a dangerous status of lying near the bed edge.

In addition, when any one of the pressure-sensitive sensing zones 200b, 200c, 200d, 200e and 200f senses the pressure, the server (50) or the monitoring alert apparatus (50′) may determine, according to a patient status signal, that the patient is probably on the point of dropping from the bed or the patient intends to leave the bed, the control terminal sends a warning signal to the alert device, and displays patient information on an LED panel of the nurse station. In this case, the alert device shows a warning red lamp and emits buzz sound, to indicate that the patient is currently in a dangerous status. After receiving the signal, nursing staff can timely go to the ward to handle the situation. Likewise, when the pressure-sensitive sensing zones 200b and 200d, or 200c and 200e, or 200d and 200f, or 200e and 200f simultaneously sense the pressure, it also indicates that the patient is currently in a dangerous status.

In some embodiments, the signal converter (30), or the server (50), or the monitoring alert apparatus (50′) measures the time duration of each detected pressure contact by e.g., measuring the time duration of each pressure-sensitive sensing zones 200b, 200c, 200d, 200e and 200f sensing pressure. The signal converter (30) may measure the duration of each pressure contact based on the electrical signals it received from the different pressure-sensitive sensing zones 200b, 200c, 200d, 200e and 200f. The server (50), or the monitoring alert apparatus (50′) may measure the time duration of each detected pressure contact based on the patient status signals it receives from the signal converter (30) and signal transmitter (40). When the duration of any of the pressure contacts exceeds a specified threshold time, the server (50) or the monitoring alert apparatus (50′) generates an alert. This alert can be used to notify a caretaker to reposition the patient to avoid any prolonged pressure contact. This provides an effective and inexpensive way to prevent, manage, and treat pressure ulcers or bedsores in a bed ridden patient.

If the nursing staff fail to timely handle the situation after receiving the signal indicating that the patient is in a dangerous status, the server (50) or the monitoring alert apparatus (50′) may send the corresponding warning signal to the nursing staff holding the alert device at regular intervals, till the nursing staff arrive on site to handle the situation and presses a warning lifting button. The warning lifting button may be configured near the sickbed, on the signal converter, or on the calling device in the ward. After the alarm is lifted, a signal is returned to the server (50) or the monitoring alert apparatus (50′), and then the server (50) stops sending the warning signal and records the warning lifting time.

Further, a few nursing staffs need to take care of multiple patients, or the nursing staff are probably on the move or do not stay on a set position for a long time. Such cases often happen in reality. A ZigBee wireless transmission technology with low power consumption may be used to implement the alert device (60) as a small and portable device, such that the nursing staff can know the patient's status at any time, and thus can timely go to the ward for inspection or give proper assistance.

When the nursing staff need to take charge of a large nursing area, a repeater (not shown in the figure) for receiving patient status signals from pressure-sensitive sensor pads (20) of multiple beds (10) and then transmitting the signals to the alert device (60) may be additionally disposed. In addition, an alert device (60) may have both signal sending and receiving functions, that is, an alert device (60) may also function as a repeater for the alert devices. Thus, the effective application range of the device can be enlarged, and the loss of an important signal can be avoided.

As mentioned, in some embodiments, the pressure-sensitive sensor pads (20) is equipped with an air pump to apply alternating air pressure to the patient to alleviate bed sores or pressure ulcers. In some of these embodiments, the air pressure of the pressure-sensitive sensing zones (200) are independently controlled based on the patient signals. For example, in some embodiments, the server (50) or the monitoring alert apparatus (50′) or an alert device (60) may use the received patient status signals to determine which of the pressure-sensitive sensing zones (200) is in danger of having a prolonged pressure contact (e.g., when the duration of a pressure contact is longer than a threshold) and accordingly activate alternating air pressure for that zone.

In some embodiments, the server (50) or the monitoring alert apparatus (50′) may graphically display various patient status at a display unit. The server or the monitoring alert apparatus derive these various patient statuses by interpreting the patient status signals, including those generated by the pressure-sensitive sensor pad (20). In some embodiments, the server (50) or the monitoring alert apparatus (50′) also receive and interpret patient status signals generated by other instruments, such as an oximeter, a blood pressure monitor, brief wetness monitor, and/or a glucose monitor. In some embodiments, in addition to the pressure sensors, the pressure-sensitive sensor pad (20) is also equipped with sensors that monitors the patient's heartbeat, breathing, blood pressure, and/or other statuses.

The server (50) or the monitoring alert apparatus (50′) may use these patient status signals to generate and display patient statuses such as reposition alert, falling alert, heart rate, breathing rate, brief wetness, etc.

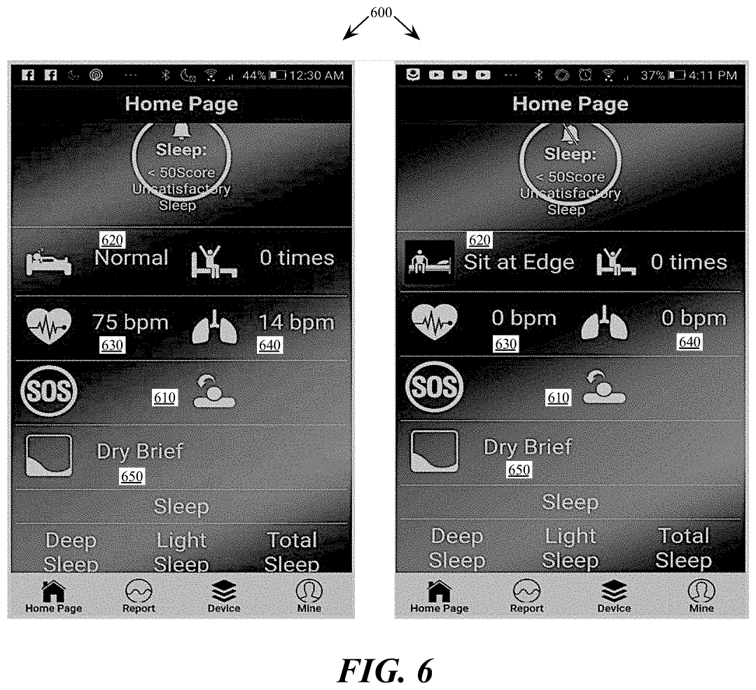

FIG. 6 illustrates an example display unit that shows the various patient status generated by the patient monitoring system. The display is generated by the server (50) or the monitoring alert apparatus (50′) based on the patent status signals received from the pressure-sensitive sensor pad (20) and other instruments. As illustrated, the display is shown at a display unit (600). It shows a reposition alert (610), a falling alert (620), a heart rate indicator (630), a breathing rate indicator (640), a brief wetness indicator (650). The reposition alert (610) is generated based on the duration of pressure contacts detected at the pressure-sensitive sensor pad (20). The falling alert (620) may be generated based on a sleep safety assessment performed based on pressure changes that are detected at different pressure-sensitive sensing zones (200) according to Table 1. The heart rate indicator (630) may be generated based on data provided by a blood pressure monitor, the breathing rate indicator (640) may be generated based on data provided by an oximeter. The brief wetness indicator (650) may be generated based on data provided by a wetness detection system, a description of which can be found in U.S. Pat. No. 10,376,422 issued on Aug. 13, 2019, titled “system and method for detecting wetness of article used by a care-receiver”.

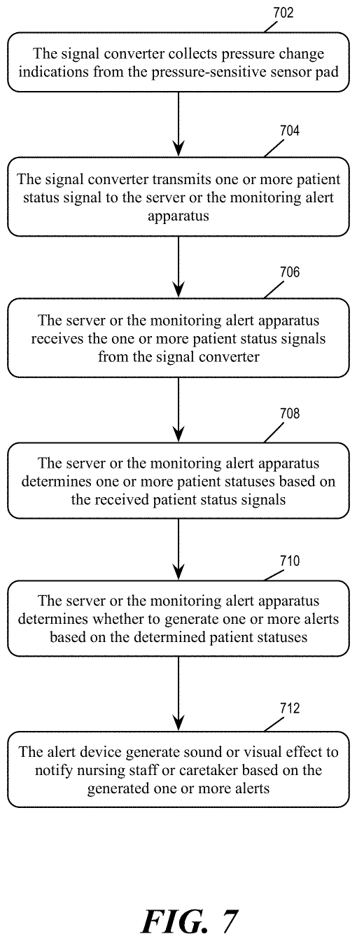

Refer to FIG. 7, which is a schematic flowchart of operations of a patient monitoring system according to the present invention. In some embodiments, one or more processing units (e.g., processor) of the computing devices implementing the signal converter (30), the server (50), and/or the monitoring alert apparatus (50′) perform the process 700 by executing instructions stored in a computer readable medium. The process 700 is illustrated as a collection of blocks in a logical flow chart, which represents a sequence of operations that can be implemented in hardware, software, or a combination thereof. In the context of software, the blocks represent computer-executable instructions that, when executed by one or more processors, perform the recited operations. Generally, computer-executable instructions may include routines, programs, objects, components, data structures, and the like, that perform particular functions or implement particular abstract data types. The order in which the operations are described is not intended to be construed as a limitation, and any number of the described blocks can be combined in any order and/or in parallel to implement the process.

At block 702, the signal converter (30) collects pressure change indications from the pressure-sensitive sensor pad (20). The pressure change indications are relayed to the signal converter as electrical signals from the different pressure-sensitive sensing zones.

At block 704, the signal converter (30) transmits one or more patient status signals to the server (50) or the monitoring alert apparatus (50′). The patient status signals are generated based on the electrical signals received from the pressure-sensitive sensor pad (20).

At block 706, the server (50) or the monitoring alert apparatus (50′) receives the one or more patient status signals from the signal converter (30). The server (50) or the monitoring alert apparatus (50′) may also receive patient status signals from other patient monitoring instruments, such as monitors for blood oxygen level, breathing rates, heart rates, blood pressure, brief wetness, etc. These instruments may include third party stand-alone devices. These instruments may also include built-in sensors of the pressure-sensitive sensor pad (20).

At block 708, the server (50) or the monitoring alert apparatus (50′) determines one or more patient statuses based on the received patient status signals. These patient statuses may include sleep safety assessment (based on the patient position), durations of pressure contacts, blood oxygen level, breathing rates, heart rates, blood pressure, brief wetness, etc. The sleep safety assessment and the duration of pressure contacts may be determined based on patient status signals that show pressure changes or pressure contacts at the different pressure-sensitive sensing zones (200) of the pressure-sensitive sensor pad (20). For example, the monitoring alert apparatus (50′) may measure the duration of each detected pressure contact. The patient status may be displayed at a display unit of the server (50) or the monitoring alert apparatus (50′). The patient status may also be displayed at a mobile device.

At block 710, the server (50) or the monitoring alert apparatus (50′) determines whether to generate one or more alerts based on the determined patient statuses. These alerts may include a reposition alert (610) and a falling alert (620). For example, the server (50) may generate the reposition alert if the duration of a pressure contact exceed a timing threshold. The server may also generate a falling alert if the sleep safety assessment indicates a patient position that is considered dangerous or abnormal according to Table 1.

At block 712, the alert devices (60) generate sound or visual effect to notify nursing staff or caretaker based on the generated one or more alerts. In some embodiments, if all of the patient statuses are normal, the server (50) may not generate any alert for any of the alert devices. The server (50) or the monitoring alert apparatus (50′) may transmit the generated alert(s) to the alert devices (60). In some embodiments, the server (50) or the monitoring alert apparatus (50′) may serve as an alert device.

In some embodiments, when performing the operations described in block 702, the signal converter also maps the electrical signals from the different pressure-sensitive sensing zones into a patient position (such as lying flat, sitting up, lying down, or lying near the bed edge) based on Table 1 above. The patient position is in turn used by the signal converter (30) as a sleep safety assessment to determine whether the patient is in a normal or abnormal status. For example, zones 200a, 200b, and 200c simultaneously sensing a pressure change maps to a patient position of lying flat, which is a normal status; zones 200a and 200b simultaneously sensing a pressure change maps to a patient position of sitting up, which is an abnormal status; zones 200b and 200c simultaneously sensing a pressure change maps to a patient position of sitting on a bed edge, which is an abnormal status; and any one of the zones 200b, 200c, 200d, and 200e sensing a pressure change maps to a patient position of leaving the bed, which is an abnormal status. The signal converter (30) in turn transmits the detected patient position and/or the determined normal/abnormal status to the sever (50) as part of the patent status signals.

In some embodiments, the signal converter does not map the pressure change indications from the different pressure-sensitive sensing zones into patient positions, but instead reports the pressure change indications from the different zones directly to the server (50) as patient status signals. The server (50) in turn maps the pressure change indications into sleep safety assessment or patient positions according to Table 1 and to generate the falling alert (620). The server (50) may also use the pressure change indications to determine the duration of pressure contacts and to generate the reposition alert (610).

According to the above descriptions and illustrations of the embodiments, it can be confirmed that the patient monitoring system disclosed in the present invention can send a signal to inform the caregiver before the patient leaves the bed and notify the caregiver to attend the patient, so that the harm caused when the patient leaves the bed alone can be minimized, thus effectively improving the work efficiency of the caregiver. Further, the patient monitoring system disclosed in the present invention can also be connected with calling system of the hospital nurse station directly, which is not only easy to install and use, but also can reduce the installation cost.

The specific embodiments described above are only used to illustrate the features and effects of the present invention, and are not intended to limit the implementation scope of present invention. Any equivalent changes and modifications made based on the content disclosed in the present invention without departing from the spirit and technical scope of the present invention still fall within the patent scope described later. For example, in some embodiments, the signal converter (30), the alert devices (60), the server (50) or the monitoring alert apparatus (50′) are connected to the Internet, and the patient status signals generated by the pressure-sensitive sensing pad (20) are uploaded to the Internet and received by the server (50), the monitoring alert apparatus (50′), and/or the alert devices (60). The server (50), the monitoring alert apparatus (50′), and/or the alert devices (60) in turn process the received patient status signals to generate the alerts and display the various patient status to the caretakers.

### Example Electronic System

Many of the above-described features and applications are implemented as software processes that are specified as a set of instructions recorded on a computer readable storage medium (also referred to as computer readable medium). When these instructions are executed by one or more computational or processing unit(s) (e.g., one or more processors, cores of processors, or other processing units), they cause the processing unit(s) to perform the actions indicated in the instructions. Examples of computer readable media include, but are not limited to, CD-ROMs, flash drives, random-access memory (RAM) chips, hard drives, erasable programmable read only memories (EPROMs), electrically erasable programmable read-only memories (EEPROMs), etc. The computer readable media does not include carrier waves and electronic signals passing wirelessly or over wired connections.

In this specification, the term “software” is meant to include firmware residing in read-only memory or applications stored in magnetic storage which can be read into memory for processing by a processor. Also, in some embodiments, multiple software inventions can be implemented as sub-parts of a larger program while remaining distinct software inventions. In some embodiments, multiple software inventions can also be implemented as separate programs. Finally, any combination of separate programs that together implement a software invention described here is within the scope of the present disclosure. In some embodiments, the software programs, when installed to operate on one or more electronic systems, define one or more specific machine implementations that execute and perform the operations of the software programs.

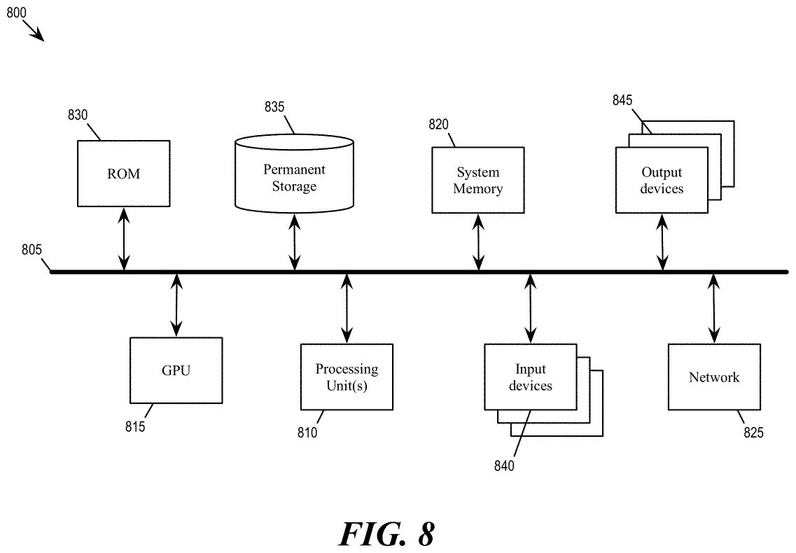

FIG. 8 conceptually illustrates an electronic system 800 with which some embodiments of the present disclosure are implemented. The electronic system 800 may be a computer (e.g., a desktop computer, personal computer, tablet computer, etc.), phone, PDA, or any other sort of electronic device. Such an electronic system includes various types of computer readable media and interfaces for various other types of computer readable media. Electronic system 800 includes a bus 805, processing unit(s) 810, a graphics-processing unit (GPU) 815, a system memory 820, a network 825, a read-only memory 830, a permanent storage device 835, input devices 840, and output devices 845.

The bus 805 collectively represents all system, peripheral, and chipset buses that communicatively connect the numerous internal devices of the electronic system 800. For instance, the bus 805 communicatively connects the processing unit(s) 810 with the GPU 815, the read-only memory 830, the system memory 820, and the permanent storage device 835.

From these various memory units, the processing unit(s) 810 retrieves instructions to execute and data to process in order to execute the processes of the present disclosure. The processing unit(s) may be a single processor or a multi-core processor in different embodiments. Some instructions are passed to and executed by the GPU 815. The GPU 815 can offload various computations or complement the image processing provided by the processing unit(s) 810.

The read-only-memory (ROM) 830 stores static data and instructions that are used by the processing unit(s) 810 and other modules of the electronic system. The permanent storage device 835, on the other hand, is a read-and-write memory device. This device is a non-volatile memory unit that stores instructions and data even when the electronic system 800 is off. Some embodiments of the present disclosure use a mass-storage device (such as a magnetic or optical disk and its corresponding disk drive) as the permanent storage device 835.

Other embodiments use a removable storage device (such as a floppy disk, flash memory device, etc., and its corresponding disk drive) as the permanent storage device. Like the permanent storage device 835, the system memory 820 is a read-and-write memory device. However, unlike storage device 835, the system memory 820 is a volatile read-and-write memory, such a random access memory. The system memory 820 stores some of the instructions and data that the processor uses at runtime. In some embodiments, processes in accordance with the present disclosure are stored in the system memory 820, the permanent storage device 835, and/or the read-only memory 830. For example, the various memory units include instructions for processing multimedia clips in accordance with some embodiments. From these various memory units, the processing unit(s) 810 retrieves instructions to execute and data to process in order to execute the processes of some embodiments.

The bus 805 also connects to the input and output devices 840 and 845. The input devices 840 enable the user to communicate information and select commands to the electronic system. The input devices 840 include alphanumeric keyboards and pointing devices (also called “cursor control devices”), cameras (e.g., webcams), microphones or similar devices for receiving voice commands, etc. The output devices 845 display images generated by the electronic system or otherwise output data. The output devices 845 include printers and display devices, such as cathode ray tubes (CRT) or liquid crystal displays (LCD), as well as speakers or similar audio output devices. Some embodiments include devices such as a touchscreen that function as both input and output devices.

Finally, as shown in FIG. 8, bus 805 also couples electronic system 800 to a network 825 through a network adapter (not shown). In this manner, the computer can be a part of a network of computers (such as a local area network (“LAN”), a wide area network (“WAN”), or an Intranet, or a network of networks, such as the Internet. Any or all components of electronic system 800 may be used in conjunction with the present disclosure.

Some embodiments include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra-density optical discs, any other optical or magnetic media, and floppy disks. The computer-readable media may store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, many of the above-described features and applications are performed by one or more integrated circuits, such as application specific integrated circuits (ASICs) or field programmable gate arrays (FPGAs). In some embodiments, such integrated circuits execute instructions that are stored on the circuit itself. In addition, some embodiments execute software stored in programmable logic devices (PLDs), ROM, or RAM devices.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium,” “computer readable media,” and “machine readable medium” are entirely restricted to tangible, physical objects that store information in a form that is readable by a computer. These terms exclude any wireless signals, wired download signals, and any other ephemeral signals.

While the present disclosure has been described with reference to numerous specific details, one of ordinary skill in the art will recognize that the present disclosure can be embodied in other specific forms without departing from the spirit of the present disclosure. In addition, a number of the figures (including FIG. 7) conceptually illustrate processes. The specific operations of these processes may not be performed in the exact order shown and described. The specific operations may not be performed in one continuous series of operations, and different specific operations may be performed in different embodiments. Furthermore, the process could be implemented using several sub-processes, or as part of a larger macro process. Thus, one of ordinary skill in the art would understand that the present disclosure is not to be limited by the foregoing illustrative details, but rather is to be defined by the appended claims.

What is claimed is:

1. A patient monitoring system, comprising:
a pressure-sensitive sensor pad, comprising an upper conductive layer, a bottom conductive layer and an insulating layer arranged therebetween, wherein the upper conductive layer and the bottom conductive layer can generate an electrical signal due to a pressure change;
a signal converter, electrically connected to the pressure-sensitive sensor pad, and used to receive the electrical signal sent by the pressure-sensitive sensor pad and to perform operational analysis to produce a corresponding patient status signal; and
a signal transmitter, electrically connected to the signal converter, and used to transmit the patient status signal from the signal converter, wherein the pressure-sensitive sensor pad is further divided into at least one or a plurality of pressure-sensitive sensing zones, wherein the at least one or the plurality of pressure-sensitive sensing zones produces electrical signals due to a foreign force or pressure change, the electrical signals being the same or varying from each other, or some of them being the same.

2. The patient monitoring system of claim 1, wherein the at least one or the plurality of pressure-sensitive sensing zones has the same or different sensing areas, or some of them have the same sensing areas.

3. The patient monitoring system of claim 2, wherein the insulating layer is further provided with multiple pierced regions.

4. The patient monitoring system of claim 1, further comprising a server, used to receive the patient status signal sent by the signal transmitter, wherein the server further comprises a display unit used to store and/or display information corresponding to the patient status signal.

5. The patient monitoring system of claim 1, further comprising at least one alert device used to send out a warning signal.

6. The patient monitoring system of claim 5, wherein the warning signal is presented in at least one of the following manners: sound, an image, vibration, light, and a digital signal.

7. The patient monitoring system of claim 5, wherein the alert device is at least one of a wireless BB call, a mobile phone, a buzzer, an alarm lamp, and an audio alarm.

8. The patient monitoring system of claim 5, wherein when the patient is in a dangerous status, the alert device unceasingly sends out the warning signal at regular intervals, till the warning is lifted.

9. The patient monitoring system of claim 1, wherein the signal transmitter transmits a signal via a wireless transmission manner such as WIFI, ZigBee, or Bluetooth.

10. The patient monitoring system of claim 1, wherein the pressure-sensitive sensor pad is arranged in a bed, the bed further comprises an upper cushion and a bottom cushion, and the pressure-sensitive sensor pad is placed on the upper cushion or between the upper cushion and the bottom cushion.

11. A computer-implemented method comprising:
receiving, at a computing device, from a pressure-sensitive sensor pad, a first set of one or more patient status signals that are generated due to pressure changes at one or more of a plurality of pressure-sensitive sensing zones that populate the pressure-sensitive sensor pad;
detecting one or more pressure contacts between a patient and the pressure-sensitive sensor pad based on the first set of patient status signals;
measuring a time duration for each detected pressure contact; and
generating an alert when a measured time duration of a detected pressure contact exceeds a threshold time duration.

12. The computer-implemented method of claim 11, further comprising reporting a heart rate, a breathe rate, and a brief dryness based on a second set of patient status signals that are generated by one or more instruments other than the pressure-sensitive sensor pad.

13. The computer-implemented method of claim 11, further comprising:
identifying a patient position based on the first set of patient status signals; and
reporting a sleep safety assessment based on the identified patient position.

14. The computer-implemented method of claim 11, wherein the pressure-sensitive sensor pad comprises an upper conductive layer, a bottom conductive layer and an insulating layer arranged therebetween, wherein the insulating layer comprises a set of pierced regions that partition the pressure-sensitive sensor pad into a plurality of pressure-sensitive sensing zones, wherein the upper conductive layer and the bottom conductive layer is configured to generate electrical signals due to pressure changes at one or more of the plurality of pressure-sensitive sensing zones.

15. The computer-implemented method of claim 11, wherein the pressure-sensitive sensor pad comprises an air pump to provide alternating air pressure in the pressure-sensitive sensing zones, wherein the computer-implemented method further comprises using the received patient status signals to identify a pressure-sensitive sensing zone having a time duration of a pressure contact that exceed a threshold and activating alternating air pressure for that zone.

16. A computer program product comprising:
one or more non-transitory computer-readable storage devices and program instructions stored on at least one of the one or more non-transitory storage devices, the program instructions executable by a processor, the program instructions comprising sets of instructions for:
receiving, at a computing device, from a pressure-sensitive sensor pad, a first set of one or more patient status signals that are generated due to pressure changes at one or more of a plurality of pressure-sensitive sensing zones that populate the pressure-sensitive sensor pad;
detecting one or more pressure contacts between a patient and the pressure-sensitive sensor pad based on the first set of patient status signals;
measuring a time duration for each detected pressure contact; and
generating an alert when a measured time duration of a detected pressure contact exceeds a threshold time duration.

17. The computer program product of claim 16, wherein the program instructions further comprising sets of instructions for reporting a heart rate, a breathe rate, and a brief dryness based on a second set of patient status signals that are generated by one or more instruments other than the pressure-sensitive sensor pad.

18. The computer program product of claim 16, wherein the program instructions further comprising sets of instructions for:
identifying a patient position based on the first set of patient status signals; and
reporting a sleep safety assessment based on the identified patient position.

19. The computer program product of claim 16, wherein the pressure-sensitive sensor pad comprises an upper conductive layer, a bottom conductive layer and an insulating layer arranged therebetween, wherein the insulating layer comprises a set of pierced regions that partition the pressure-sensitive sensor pad into a plurality of pressure-sensitive sensing zones, wherein the upper conductive layer and the bottom conductive layer is configured to generate electrical signals due to pressure changes at one or more of the plurality of pressure-sensitive sensing zones.

20. The computer program product of claim 16, wherein the pressure-sensitive sensor pad comprises an air pump to provide alternating air pressure in the pressure-sensitive sensing zones, wherein the program instructions further comprises sets of instructions using the received patient status signals to identify a pressure-sensitive sensing zone having a time duration of a pressure contact that exceed a threshold and activating alternating air pressure for that zone.
